(19) United States
(12) Patent Application Publication
(10) Pub. No.: US 2026/0221235 A1
(43) Pub. Date: Jul. 30, 2026

(54) END-OF-LIFE INSTRUCTIONS

(71) Applicant: BASE SE, Ludwigshafen am Rhein (DE)

(72) Inventors: Gabriele ECKARDT, Ludwigshafen am Rhein (DE); Juergen SEITZ, Ludwigshafen am Rhein (DE)

(21) Appl. No.: 19/146,779

(22) PCT Filed: Jan. 9, 2024
(86) PCT No.: PCT/EP2024/050382

§ 371 Date: Jul. 9, 2025

(30) Foreign Application Priority Data
Jan. 9, 2023 (EP) 23150672.6

(51) Int. Cl. G16C 20/20

(52) U.S. Cl. CPC G16C 20/20

(57) ABSTRACT

Disclosed are methods, apparatuses, systems and end-of-life instructions configured to control one or more end-of-life product(s) to recover at least one chemical material from the one or more end-of-life product(s) as well as chemical product(s) associated with end-of-life instructions configured to control one or more end-of-life product(s) for recovery of at least one chemical material from the one or more end-of-life product(s), instruction consuming services, instruction providing services, data providing services, data consuming services configured to control one or more end-of-life product(s) for recovery of at least one chemical material from the one or more end-of-life product(s).

### TECHNICAL FIELD

Disclosed are methods, apparatuses, systems and end-of-life instructions configured to control one or more end-of-life product(s) to recover at least one chemical material from the one or more end-of-life product(s) as well as chemical product(s) associated with end-of-life instructions configured to control one or more end-of-life product(s) for recovery of at least one chemical material from the one or more end-of-life product(s), instruction consuming services, instruction providing services, data providing services, data consuming services configured to control one or more end-of-life product(s) for recovery of at least one chemical material from the one or more end-of-life product(s).

### TECHNICAL BACKGROUND

Chemical products are used in diverse applications and end up in multiple supply chains. End-of-life products containing chemical products are not easily available to chemical producers.

This hampers reliably refeed used products into chemical production and the built up of circular value chains.

### SUMMARY

In one aspect disclosed is a method for controlling one or more end-of-life product(s) to recover at least one chemical material from the one or more end-of-life product(s), wherein the one or more end-of-life product(s) are produced based on at least one chemical product, the method comprising the steps:
providing, based on at least one end-of-life trigger, at least one end-of-life instruction associated with the chemical product, wherein the at least one end-of-life trigger is associated with the at least one chemical product and at least one chemical product identifier;
generating, based the at least one end-of-life instruction, control data associated with the recovery of the at least one chemical material from the one or more end-of-life product(s) produced based on at least one chemical product;
providing the control data to initialize a process for recovering the chemical material.

In another aspect disclosed is a method for controlling one or more end-of-life product(s) to recover at least one chemical material from the one or more end-of-life product(s), wherein the one or more end-of-life product(s) are produced based on at least one chemical product, wherein the one or more end-of-life product(s) are controlled via peer-to-peer communication between computing node(s) of a decentral network, the method comprising the steps:
providing, based on at least one end-of-life trigger provided by one or more computing node(s) of the decentral network, at least one end-of-life instruction associated with the chemical product, wherein the at least one end-of-life trigger is associated with the at least one chemical product and at least one chemical product identifier;
generating, based on at least one end-of-life instruction, by one or more computing node(s) of the decentral network control data associated with the recovery of the at least one chemical material from the one or more end-of-life product(s) produced based on at least one chemical product;
providing the control data to one or more computing node(s) of the decentral network, wherein the computing nodes are configured to initialize a process for recovering the chemical material.

In another aspect disclosed is an apparatus for controlling one or more end-of-life product(s) to recover at least one chemical material from the one or more end-of-life product(s), wherein the one or more end-of-life product(s) are produced based on at least one chemical product, the apparatus being configured to:
providing, based on at least one end-of-life trigger, at least one end-of-life instruction associated with the chemical product, wherein the at least one end-of-life trigger is associated with the at least one chemical product and at least one chemical product identifier;
generating, based on at least one end-of-life instruction, control data associated with the recovery of the at least one chemical material from the one or more end-of-life product(s) produced based on at least one chemical product;
providing the control data to initialize a process for recovering the chemical material.

In another aspect disclosed is an apparatus for controlling one or more end-of-life product(s) to recover at least one chemical material from the one or more end-of-life product(s), wherein the one or more end-of-life product(s) are produced based on at least one chemical product, wherein the one or more end-of-life product(s) are controlled via peer-to-peer communication between computing node(s) of a decentral network, the apparatus being configured to:
providing, based on at least one end-of-life trigger provided by one or more computing node(s) of the decentral network, at least one end-of-life instruction associated with the chemical product, wherein the at least one end-of-life trigger is associated with the at least one chemical product and at least one chemical product identifier;
generating, based on the at least one end-of-life instruction, by one or more computing node(s) of the decentral network control data associated with the recovery of the at least one chemical material from the one or more end-of-life product(s) produced based on at least one chemical product;
providing the control data to one or more computing node(s) of the decentral network, wherein the computing nodes are configured to initialize a process for recovering the chemical material.

In another aspect disclosed is an apparatus for controlling one or more end-of-life product(s) to recover at least one chemical material from the one or more end-of-life product(s), wherein the one or more end-of-life product(s) are produced based on at least one chemical product, the apparatus comprising:
one or more computing node(s) and one or more computer-readable media having thereon computer-executable instructions that are structured such that, when executed by the one or more computing node(s), cause the apparatus to perform the following steps:
providing, based on at least one end-of-life trigger, at least one end-of-life instruction associated with the chemical product, wherein the at least one end-of-life trigger is associated with the at least one chemical product and at least one chemical product identifier;
generating, based on at least one end-of-life instruction, control data associated with the recovery of the at least one chemical material from the one or more end-of-life product(s) produced based on at least one chemical product;
providing the control data to initialize a process for recovering the chemical material.

In another aspect disclosed is an apparatus for controlling one or more end-of-life product(s) to recover at least one chemical material from the one or more end-of-life product(s), wherein the one or more end-of-life product(s) are produced based on at least one chemical product, wherein the one or more end-of-life product(s) are controlled via peer-to-peer communication between computing node(s) of a decentral network, the apparatus comprising:
one or more computing node(s) and one or more computer-readable media having thereon computer-executable instructions that are structured such that, when executed by the one or more computing node(s), cause the apparatus to perform the following steps:
   providing, based on at least one end-of-life trigger provided by one or more computing node(s) of the decentral network, at least one end-of-life instruction associated with the chemical product, wherein the at least one end-of-life trigger is associated with the at least one chemical product and at least one chemical product identifier;
   generating, based on the at least one end-of-life instruction, by one or more computing node(s) of the decentral network control data associated with the recovery of the at least one chemical material from the one or more end-of-life product(s) produced based on at least one chemical product;
   providing the control data to one or more computing node(s) of the decentral network, wherein the computing nodes are configured to initialize a process for recovering the chemical material.

In another aspect disclosed is a system for controlling one or more end-of-life product(s) to recover at least one chemical material from the one or more end-of-life product(s), wherein the one or more end-of-life product(s) are produced based on at least one chemical product, the system comprising:
an apparatus for controlling one or more end-of-life product(s) to recover at least one chemical material from the one or more end-of-life product(s),
optionally a plant operator of a chemical and/or physical treatment plant configured to receive the control data to initialize a process for recovering the chemical material, wherein the operator of the chemical and/or physical treatment plant may be associated with the chemical product producer;
optionally a chemical and/or physical treatment plant for treating the end-of-life product or part(s) thereof to recover the chemical material, wherein the chemical and/or physical treatment plant may be associated with the chemical product producer.

In another aspect disclosed is a system for controlling one or more end-of-life product(s) to recover at least one chemical material from the one or more end-of-life product(s), wherein the one or more end-of-life product(s) are produced based on at least one chemical product, the system comprising:
a trigger provider configured to generate at least one end-of-life trigger based on at least one end-of-life event associated with one or more end-of-life product(s), wherein the at least one end-of-life trigger is associated with the at least one chemical product and at least one chemical product identifier;
an instructions provider configured to provide, based on at least one end-of-life trigger, at least one end-of-life instruction associated with the chemical product;
an instructions executor configured to generate, based on at least one end-of-life instruction, control data associated with the recovery of the at least one chemical material from the one or more end-of-life product(s) produced based on at least one chemical product;
a control data provider configured to provide the control data to initialize a process for recovering the chemical material;
optionally a plant operator of a chemical and/or physical treatment plant configured to receive the control data to initialize the process for recovering the chemical material, wherein the operator of the chemical and/or physical treatment plant may be associated with the chemical product producer;
optionally a chemical and/or physical treatment plant for treating the end-of-life product or part(s) thereof to recover the chemical material, wherein the chemical and/or physical treatment plant may be associated with the chemical product producer.

In another aspect disclosed are end-of-life instruction(s) configured to control one or more end-of-life product(s) for recovery of at least one chemical material from the one or more end-of-life product(s) according to the methods disclosed herein or by the apparatuses or systems disclosed herein.

In another aspect disclosed are one or more chemical product(s) associated with at least one end-of-life instruction configured to or used to control one or more end-of-life product(s) for recovery of at least one chemical material from the one or more end-of-life product(s) according to the methods disclosed herein or by the apparatuses or systems disclosed herein.

In another aspect disclosed is a system including one or more chemical product(s) and at least one end-of-life instruction configured to or used to control one or more end-of-life product(s) for recovery of at least one chemical material from the one or more end-of-life product(s) according to the methods disclosed herein or the apparatuses or systems disclosed herein, wherein the chemical product(s) are associated with the at least one end-of-life instruction.

In another aspect disclosed are instruction consuming service(s) and/or instruction providing service(s) configured to control one or more end-of-life product(s) for recovery of at least one chemical material from the one or more end-of-life product(s) according to the methods disclosed herein or the apparatuses or systems disclosed herein.

In another aspect disclosed are instruction consuming service(s) configured to generate at least one end-of-life trigger and/or to receive at least one end-of-life instruction based on at least one end-of-life trigger, wherein the at least one end-of-life trigger and/or the at least one end-of-life instruction may be configured to or used to control one or more end-of-life product(s) for recovery of at least one chemical material from the one or more end-of-life product(s) according to the methods disclosed herein or the apparatuses or systems disclosed herein.

In another aspect disclosed are instruction providing service(s) configured to receive at least one end-of-life trigger from instruction consuming service(s) and/or to send at least one end-of-life instruction based on at least one end-of-life trigger to instruction consuming service(s) configured to control one or more end-of-life product(s) for recovery of at least one chemical material from the one or more end-of-life product(s) according to the methods disclosed herein or the apparatuses or systems disclosed herein.

In another aspect disclosed are instruction providing service(s) configured to provide end-of-life instructions or instruction consuming service(s) configured to consume end-of-life instructions, wherein the at least one end-of-life trigger and/or the at least one end-of-life instruction may be configured to or used to control one or more end-of-life product(s) for recovery of at least one chemical material from the one or more end-of-life product(s) according to the methods disclosed herein or the apparatuses or systems disclosed herein.

In another aspect disclosed are data providing service(s) or data consuming service(s) configured to control one or more end-of-life product(s) to recover at least one chemical material from the one or more end-of-life product(s) according to the methods disclosed herein or the apparatuses or systems disclosed herein.

In another aspect disclosed is the use of the control data provided according to the methods disclosed herein or by the apparatuses or systems disclosed herein to recover chemical material and/or to refeed chemical material into one or more chemical production process(es).

In another aspect the present disclosure relates to a computer element, such as a computer program or computer readable medium, with instructions, which when executed on one or more computing node(s) of a decentral network are configured to carry out the steps of the method(s) disclosed herein or are configured to be carried out by the apparatuses or systems disclosed herein.

Any disclosure, embodiments and examples described herein relate to the methods, the systems, apparatuses, chemical products, chemical product passports, end-of-life instructions, consuming services, providing services and computer elements lined out above and below. Advantageously, the benefits provided by any of the embodiments and examples equally apply to all other embodiments and examples.

### EMBODIMENTS

To improve refeed of used products into chemical production networks, products with valuable chemical product content may be tracked and traced via decentral networks. To reliably use and refeed suitable end-of-life products or components thereof into the chemical infrastructure, end-of-life instructions are proposed. The end-of-life instructions may be provided to end-of-life product users via the decentral network. This way the end-of-life process from collecting of end-of-life products to treating end-of life products and reusing chemical material may be controlled by chemical producers to reliably refeed chemical materials back into their chemical production chains. Circular value chains may be enabled to reduce the environmental impact of material flows.

In the following, embodiments of the present disclosure will be outlined by ways of examples. It is to be understood that the present disclosure is not limited to said embodiments and/or examples.

The chemical product may be produced by a chemical product producer. The chemical product may be the physical entity of the produced chemical product. Chemical product data, in particular one or more end-of-life instruction(s), may be associated with the chemical product. The chemical product data may relate to a digital twin of the chemical product including e.g. one or more end-of-life instruction(s). The chemical product data may be collected before, during or after production of the chemical product. The chemical product data, in particular one or more end-of-life instruction(s), may be generated by the chemical product producer. The chemical product data may be associated with the produced chemical product or properties of the produced chemical product. The chemical product data, in particular one or more end-of-life instruction(s), may be provided for access through the decentral network by the participant node associated with the chemical product producer. The chemical product data, in particular one or more end-of-life instruction(s), may be provided for access through the decentral network by the data providing service of the chemical product producer.

The chemical product may be used to produce the product. The product produced using the chemical product may be an intermediate product or an end product. The product may be produced through a production including one or more intermediate production steps. The product may be used through the lifecycle of a product. The chemical product, the intermediate product and/or the end product may be related to one or more decentral identifier(s) signifying the digital twin of the physical entity of such product entities. The decentral identifier(s) may be digital identifier(s) of or for the decentral network. The decentral identifier(s) may be a digital identifier provided to the decentral network and computing nodes associated with participants of the decentral network. The decentral identifier(s) may hence signify physical entities of products for participant node(s) of the decentral network. The decentral identifier(s) may be linked according to the use of the physical product entities in the production chain of at least one end product.

The decentral identifier(s) may be linked according to the physical relation of physical product entities used to produce the end product. This way participant node(s) of the decentral network may be able to interpret the relation of the decentral identifier(s) corresponding to the physical relation of physical product entities.

The end-of-life product may be produced based on at least one chemical product. The end-of-life product may contain at least one chemical product. The end-of-life product may be an end product used during the lifecycle of the end product. The end-of-life product may be related to one or more decentral identifier(s) signifying the product entities used to produce the end product. The end-of-life product may be related to one or more decentral identifier(s) signifying at least one chemical product used to produce the end product. The end-of-life product may be related to one or more end-of-life instruction(s) related to the recovery of chemical material from the end-of-life product.

At least one chemical material may be recovered from the one or more end-of-life product(s). For recovery, the end-of-life product may be at least partially decomposed into its chemical and/or physical components. The end-of-life product or parts thereof may be collected and/or sorted. The end-of-life product or parts thereof may be chemically and/or physically treated. The end-of-life product or parts thereof may be chemically and/or physically treated to produce chemical material that may be refed into the chemical production process. The process for recovering the chemical material may include multiple steps, such as collecting, sorting, transporting, storing, returning or treating end-of-life product(s) or parts thereof. The process for recovering the chemical material may include returning end-of-life product(s) or parts thereof. The process for recovering the chemical material may include chemically and/or physically treating end-of-life product(s) or parts thereof.

In one embodiment the chemical material includes one or more chemical compound(s), chemical component(s) or chemical composition(s) produced from the end-of-life product or part(s) thereof. The chemical material may be recovered from the end-of-life product or part(s) thereof.

The chemical material may be traced back to the chemical product used to produce the end-of-life product or part(s) thereof. The production chain to produce the end-of-life product or part(s) thereof may include at least the chemical product produced by the chemical product producer and the chemical product used to produce the end product. The life-cycle chain of the end product may include the use to the end-of-life. The chemical material may be the chemical compound, chemical component or chemical composition to be recovered from the end-of-life product.

In another embodiment the chemical material includes one or more chemical compound(s), chemical component(s) or chemical composition(s) suitable for producing the at least one chemical product. The chemical material may be the output product of a chemical and/or physical treatment process of end-of-life product(s) or part(s) thereof. The chemical material may include or be the original chemical product compound, component or composition. The chemical material may hence be chemically equal to the chemical product. The chemical material may include the original compounds, components or compositions used to produce chemical product. The chemical material may hence be used to synthesize the chemical product. The chemical material may include one or more sub-compound(s), sub-component(s) or sub-composition(s). The chemical material may include one or more reaction product(s) of (sub-) compounds, (sub-) component(s) or (sub-) composition(s) of the chemical product. The chemical material may hence comprise reaction components used to produce the chemical product. The chemical material may include or be a chemical precursor for producing the chemical product. The chemical material may be a chemical mixture.

In another embodiment the at least one end-of-life trigger is associated with at least one decentral identifier associated with the chemical product used to produce or contained in the end-of-life product. The at least one end-of-life trigger may include the at least one decentral identifier associated with the chemical product used to produce or contained in the end-of-life product.

The at least one end-of-life trigger may be based on an end-of-life event. The end-of-life event may include, but is not limited to, the detection of an end-of-life state via sensor(s), the detection of the end-of-life state by an interaction with the end product or end-of-life product user, the determination of the end-of-life state through meta data associated with the end-of-life product such as location, the determination of the end-of-life state by predefined conditions such as timing.

The decentral identifier may be uniquely associated with the chemical product or the physical entity of the chemical product, e.g. as packaged for transportation to the consumer of the chemical product. The decentral identifier may be connected to the digital representation of chemical product data, in particular one or more end-of-life instruction(s). The data related to chemical product data may include one or more digital representation(s) pointing to chemical product data or part(s) thereof, in particular one or more end-of-life instruction(s). The digital representation may comprise at least one interface to the data providing service of the chemical product producer. The digital representation may further include at least one interface to the data consuming service of the chemical product producer. It may include an endpoint for data exchange or sharing (resource endpoint) or an endpoint for service interaction (service endpoint), that is uniquely identified via a data transaction protocol including authentication and/or authorization mechanisms. The digital representation(s) pointing to chemical product data or parts thereof, in particular one or more end-of-life instruction(s), may be uniquely associated with the decentral identifier associated with the chemical product.

At the end-of-life stage, the at least one end-of-life trigger may be generated by a trigger providing service associated with the user of the end-of-life product. The computing node configured to provide the trigger providing service may be associated with the user of the end-of-life product as participant of the decentral network. At the end-of-life stage the at least one end-of-life trigger may be provided to a trigger consuming service associated with the chemical product producer. The computing node configured to provide the trigger consuming service may be associated with the chemical producer as participant of the decentral network.

In another embodiment the at least one end-of-life instruction associated with the chemical product used to produce or contained in the end-of-life product is provided in relation with the at least one decentral identifier associated with the chemical product used to produce the end-of-life product. The decentral identifier associated with the chemical product used to produce or contained in the end-of-life product may be linked to or anchored by the decentral identifier associated with the end product or the end-of-life product. The decentral identifier associated with the chemical product used to produce the end-of-life product may be linked to the decentral identifier associated with the end product or the end-of-life product. The decentral identifier associated with the chemical product used to produce the end-of-life product may have been linked to the decentral identifier associated with the end product or the end-of-life product on production or providing of the end product. The decentral identifier associated with the end-of-life product may be associated with a physical identifier connected to the end-of-life product e.g. via QR code or RFID tag.

The at least one end-of-life instruction may be configured to initialize the process for recovering the chemical material. The at least one end-of-life instruction may be used to generate control data by providing or fetching the control data from the computing node associated with the chemical product producer or by determining the control data based on the end-of-life instructions provided by the computing node associated with the chemical product producer. The initialization of the process for recovering the chemical material may include the generating and/or providing of control data.

The at least one end-of-life instruction associated with the chemical product may be provided via the decentral network from an instructions providing service associated with the chemical product producer. The at least one end-of-life instruction associated with the chemical product may be provided via the decentral network to an instructions consuming service associated with the end-of-life product user. The at least one end-of-life instruction associated with the chemical product may be provided via the decentral network to an instructions consuming service associated with any user following the end-of-life product user of the process for recovering the chemical material.

In another embodiment the at least one end-of-life instruction associated with the chemical product is provided in response to providing the end-of-life trigger to an instructions providing service associated with an end product producer, an end product user or an end-of-life product user. Additionally or alternatively, the at least one end-of-life instruction associated with the chemical product may be provided via the decentral network from the instructions providing service associated with the end product producer, the end product user or the end-of-life product user.

In another embodiment the at least one end-of-life instruction associated with the chemical product is provided in response to providing the end-of-life trigger to an instructions providing service associated with a chemical product producer. Additionally or alternatively, the at least one end-of-life instruction associated with the chemical product may be provided via the decentral network from the instructions providing service associated with the chemical product producer.

In another embodiment the at least one end-of-life instruction associated with the chemical product is be configured to generate or provide control data for providing the end-of-life product or at least one part of the end-of-life product to a chemical and/or physical treatment plant associated with the chemical product producer. The at least one end-of-life instruction associated with the chemical product may be used to generate control data for recovering the chemical material derivable from the end-of-life product or at least one part of the end-of-life product.

In another embodiment the control data associated with one or more process step(s) of the reuse process are generated. Additionally or alternatively, the reuse process includes returning end-of-life product(s) or parts thereof to the chemical product producer and/or treating end-of-life product(s) or parts thereof by the chemical product producer.

In another embodiment the control data relates to a chemical and/or physical treatment of the end-of-life product to recover the chemical material. The control data may relate to chemical and/or physical treatment method(s) suitable for treating the end-of-life product or part(s) thereof to recover the chemical material. The control data may relate to chemical and/or physical treatment method(s) to be conducted by the chemical product producer. Additionally or alternatively, the control data may relate to a return of the end-of-life product or part(s) thereof to the chemical product producer. The control data may relate to chemical and/or physical treatment plant(s) suitable for treating the end-of-life product or part(s) thereof to recover the chemical material. The control data may relate to chemical and/or physical treatment plant(s) suitable for recovering the chemical material from the end-of-life product or part(s) thereof. The control data may relate to collecting, sorting and/or transporting of end-of-life products or part(s) thereof to recover the chemical material. The control data may relate to providing the end-of-life product or at least one part of the end-of-life product to a chemical and/or physical treatment plant associated with a chemical product producer.

The decentral network may include computing nodes associated with participants of the decentral network which may be configured to perform data transactions. The computing nodes associated with participants of the decentral network may be associated with producers or user of physical products, such as chemical product producers, intermediate product producers, end product producers, end product users or end-of-life product users. The data transactions may be based on a transaction protocol including authentication and/or authorization mechanism(s). Based on the authentication and/or authorization mechanism(s) a peerto-peer network between computing nodes associated with participants of the decentral network may be established. The end-of-life trigger and/or the end-of-life instruction may be transferred via peer-to-peer communication between computing node(s) of the decentral network. The respective nodes of the decentral network associated with the participants of the network may be configured as instruction consuming service(s), instruction providing service(s), trigger providing service(s) or trigger consuming service(s).

The one or more authentication mechanism(s) may be associated with or linked to the decentral identifier. The one or more authentication mechanism(s) associated with the decentral identifier may be provided to participant node(s). The one or more authentication mechanism(s) associated with the decentral identifier may be accessible by the data providing service and/or the data consuming service. The decentral configuration allows for more efficient use of computing resources and strengthens control by the data owners of the decentral network.

The one or more authorization mechanism(s) may include at least one authorization rule for controlling access to data under control by the data owners. The computing nodes associated with participants of the decentral network may be configured to provide data consuming service(s) and/or data providing service(s). A data providing service may be configured to provide or send data to another participant node of the decentral network. A data consuming service may be configured to ingest or receive data from another participant node of the decentral network. Providing data to a data providing service for access by a data consuming service may include indirect or direct access of the data consuming service to the data.

### BRIEF DESCRIPTION OF THE DRAWINGS

In the following, the present disclosure is further described with reference to the enclosed figures.

FIG. 1 illustrates schematically an example of a method or apparatus for providing chemical product data, end-of-life triggers and/or end-of-life instructions via a decentral network.

FIG. 2 illustrates schematically an example of a method or apparatus for providing end-of-life instructions for the chemical product used to produce the end product.

FIGS. 3a-c illustrate schematically end-of-life instructions and product passports including the chemical product passport with end-of-life instructions.

FIG. 4 illustrates schematically another example of a method or apparatus for providing end-of-life instructions for the chemical product used to produce the end product.

FIG. 5 illustrates schematically another example of a method or apparatus for providing end-of-life instructions for the chemical product used to produce the end product.

FIG. 6 illustrates schematically a flowchart of a method for initiating a process to recover the chemical material from the end-of-life product or parts thereof based on the end-of life trigger and the end-of-life instruction associated with the chemical product.

FIG. 7a, b illustrate schematically the use of end-of life triggers to provide end-of-life instruction associated with the chemical product.

FIG. 8 illustrates schematically an example of a method or apparatus for recovering the chemical material from the end-of-life product.

FIG. 9 illustrates schematically a user interface for recovering the chemical material from the end-of-life product.

### DETAILED DESCRIPTION

The following embodiments are mere examples for implementing the methods, the apparatuses, the systems, the passports or the applications disclosed herein and shall not be considered limiting.

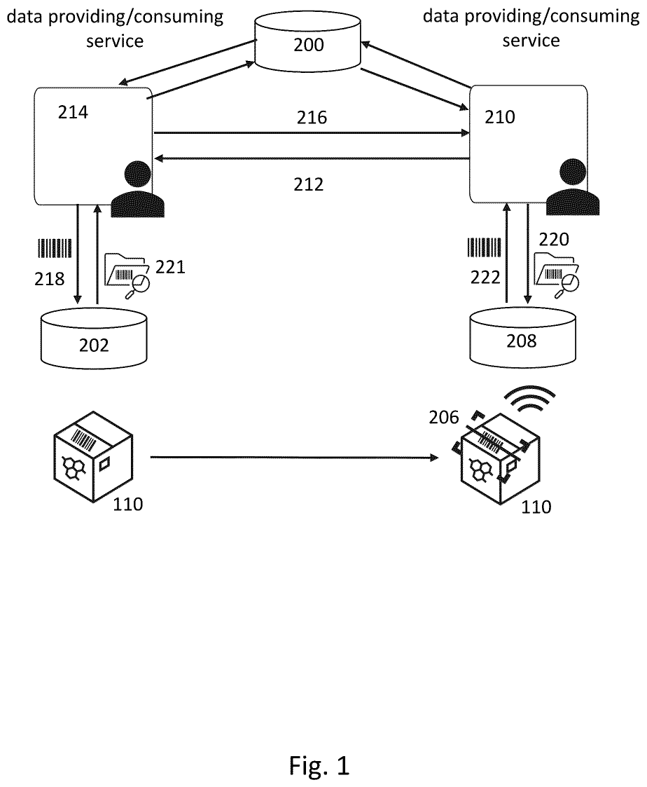

FIG. 1 illustrates schematically an example of a method or apparatus for providing chemical product data, end-of-life triggers and/or end-of-life instructions via a decentral network.

A chemical product 110 as produced by a chemical production network may be provided in association with a chemical product passport. The chemical product passport may include a chemical product identifier. The chemical product identifier may include one or more decentral identifier(s). The decentral identifier may be an identifier in the decentral network allowing for data exchange via the decentral network. Data exchange may include discovery of the decentral identifier for participants of the decentral network, authentication of participants of the decentral network and/or authorization of data transfers via a peer-to-peer communication between participants of the decentral network. The chemical product passport may include data related to the chemical product. The chemical product passport may include a digital representation of the chemical product data associated with the chemical product 110.

The chemical product passport may further include or relate to authentication and/or authorization information linked to the chemical product identifier. The authentication and/or authorization information may be provided for authentication and/or authorization of a data providing service and/or data consuming service 210, 214. The chemical product identifier may include, may be or may relate to at least one decentral identifier, that is uniquely associated with the chemical product. The decentral identifier may be connected to the digital representation of the chemical product data associated with the chemical product 110. The digital representation may include a representation for accessing the chemical product data or parts thereof. The decentral identifier may include a Universally Unique IDentifier (UUID) or a Digital IDentifier (DID). The decentral identifier may include any unique identifier uniquely associated with a data owner and/or the chemical product. The data owner may be the producer of the chemical product. Via the decentral identifier and its unique association with the data owner and/or the chemical product access to the chemical product data may be controlled by the data owner.

The chemical product passport including the digital representation of chemical product data may be stored in a decentral data base 200. The chemical product data may be stored in a data base 202 associated with the data owner, such as the producer of the chemical product 110.

The chemical product 110 may be physically delivered to a producer producing a product based on the chemical product. The chemical product may be connected with a QR-code having encoded the chemical product identifier, in particular the decentral identifier. The user of the chemical product may read the QRcode through a QR-code reader 206. The chemical product identifier may be provided to a data base 208 associated with the producer producing the product based on the chemical product 110. In other embodiments the producer producing a product based on the chemical product may retrieve the chemical product identifier through the decentral data base 200.

The data owner in this example may be the chemical product producer, any intermediate product producer producing an intermediate product based on the chemical product or any producer producing a product based on the chemical product. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service 210, 214 may comprise computer-executable instructions for accessing and/or processing data, such as chemical product data, associated with the data owner. The data providing service 210, 214 may comprise computer-executable instructions for providing and/or processing data, such as chemical product data, associated with the data owner for accessing and/or processing by the data consuming service 210, 214.

The chemical product identifier may be provided from the database 208 to the data consuming service 210 as signified by arrow 222. Based on the received chemical product identifier a request to access the chemical product data related to the chemical product identifier may be triggered by the data consuming service 210 as signified by arrow 212. The chemical product identifier may be provided to the data providing service 214 associated with or of the producer of the chemical product 110. In addition, authentication and/or authorization information may be provided. The request may be authenticated and/or authorized to access the chemical product data related to the chemical product identifier. Based on successful authorization and/or authentication access to the chemical product data related to the chemical product identifier may be granted.

For access the chemical product identifier may be provided to the data providing service 214 as signified by arrow 212. The data providing service 214 may use the received chemical product identifier to retrieve the chemical product data associated with the chemical product 110 as signified by arrows 218 and 221. The chemical product data associated with the chemical product 110 provided to the data providing service 214 may be provided to the data consuming service 210 as signified by arrow 216. The chemical product data associated with the chemical product 110 may be stored in the data base 208 associated with the user of the chemical product 110 as signified by arrow 220.

Through the chemical product identifier, in particular the decentral identifier, the chemical product data can be uniquely associated with the chemical product. Through the decentral network the chemical product data may be transferred between the producer of the chemical product and the user of the chemical product. This way the chemical product data can be shared with unique association to the chemical product and without central intermediary directly between the product supply chain players. This allows for transparency of chemical product data across the product supply chain.

Similarly, to the providing of chemical product data, end-of-life triggers and/or end-of-life instructions may be provided. Through the chemical product identifier, particularly the decentral identifier, the end-of-life triggers and/or end-of-life instructions can be uniquely associated with the chemical product. Through the decentral network the end-of-life triggers and/or end-of-life instructions may be transferred between the producer of the chemical product and the user of the end-of-life product or vice versa. This way the end-of-life triggers and/or end-of-life instructions can be shared with unique association to the chemical product and without central intermediary directly between the participants of the decentral network. Such mechanism allows for more efficient recovery of chemical products and re-use e.g. via recycling, upcycling or downcycling of chemical products contained in end products. The digital backbone structure hence enables environmentally friendly use of material. The digital backbone structure allows to reliably re-feeding used chemical products into the production of new chemical products. This way the circular use of chemical products can be enhanced and broadened to diverse set of end products made of chemical products. The digital backbone structure allows for reliably de-materializing used products to reduce environmental impact of materials not being re-usable. This way the one-way use of chemical products can be enhanced, and it can be ensured end-of-life products are treated in an environmentally friendly way.

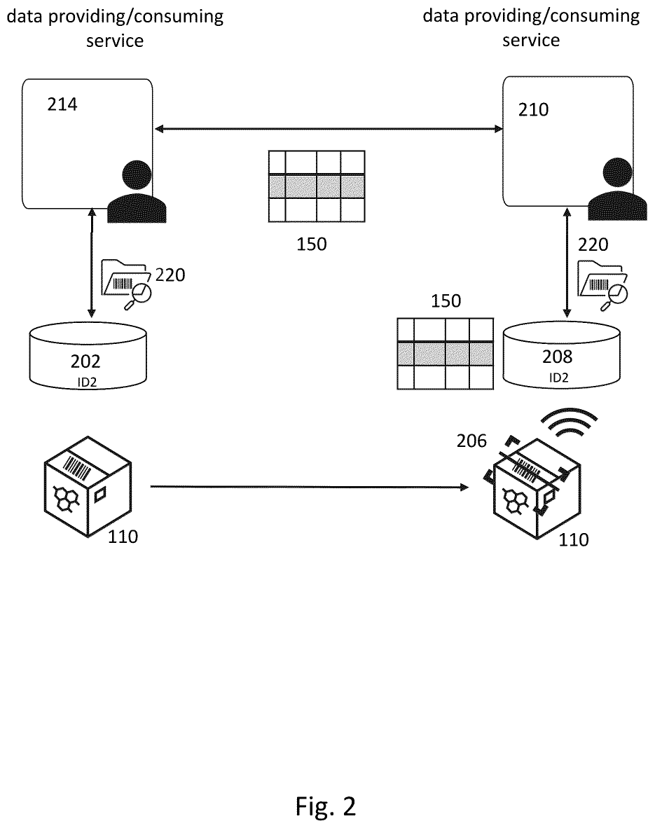

FIG. 2 illustrates schematically an example of a method or apparatus for providing end-of-life instructions 150 for the chemical product used to produce the end product via chemical product.

At end-of-life product stage, the end-of-life triggers and/or end-of-life instructions 150 may be provided and executed based on decentral network services 210, 214. Different embodiments exist and FIGS. 2-5 only illustrate examples. These shall not be considered limiting.

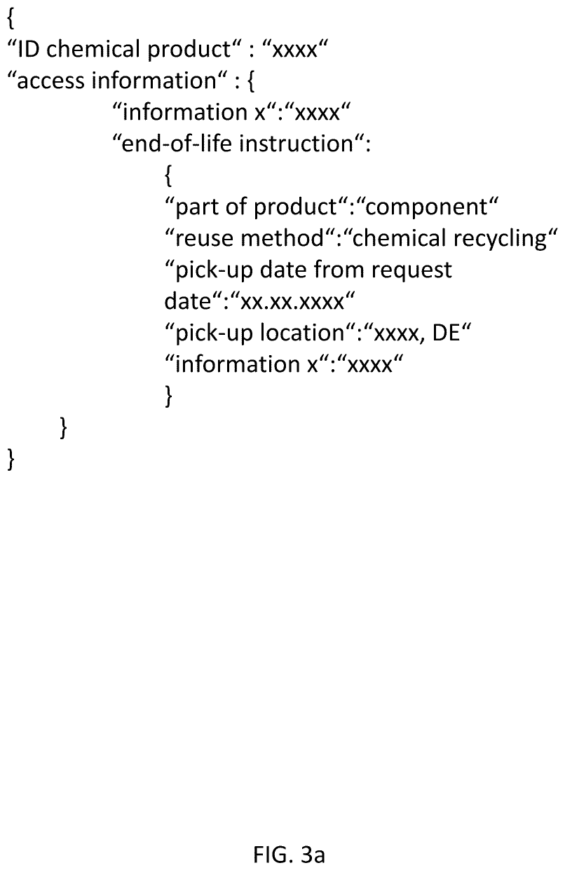

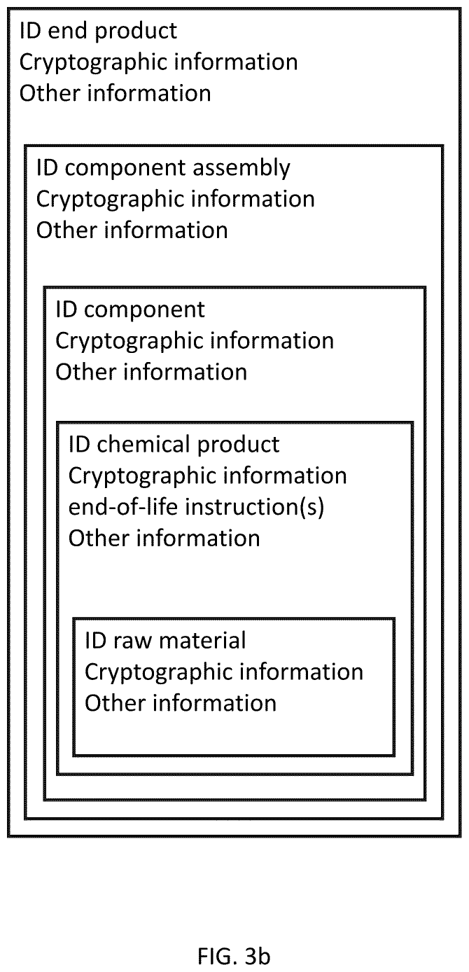

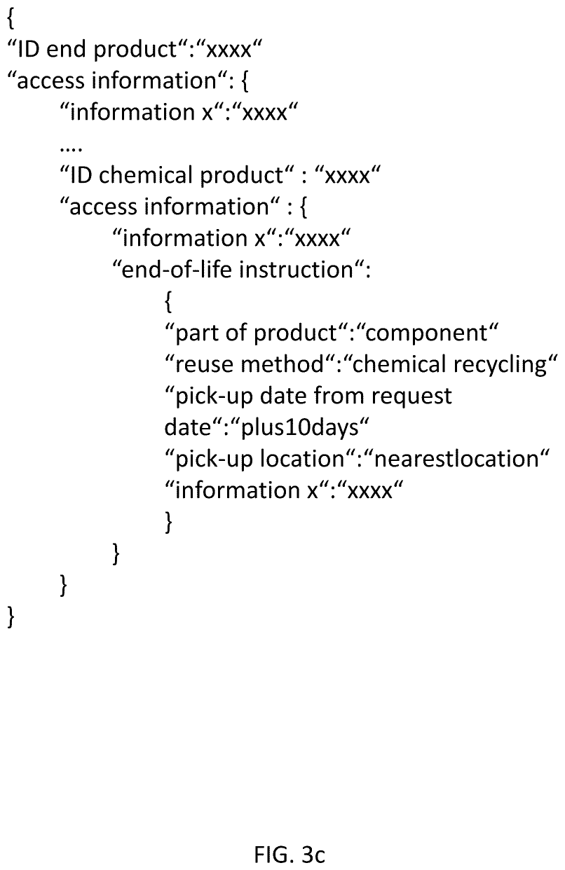

The end-of-life instructions 150 may be provided by a data providing service 214 of the chemical product producer as part of the chemical product passport. An example of such chemical product passport is described in the context of FIG. 1. The chemical product passport may be provided to all computing nodes associated with participants of the end product chain using the chemical product. This way the end-of-life instruction(s) 150 associated with the chemical product may be included into the end product passport. An example of an end product passport including the chemical product passport and the end-of-life instructions is illustrated in FIG. 3b.

As shown in FIG. 2, the end-of-life instructions 150 may be coded instructions, which may be executed based on an end-of-life trigger. The end-of-life instruction 150, when executed by a computing node associated with the participant of the decentral network, may initialize the process for recovery of the chemical material. Upon execution of the end-of-life instruction 150 by the computing node associated with the participant of the decentral network the process for recovery of the chemical material may be initialized. Participants of the decentral network 210, 214 may be the chemical product producer, the product producer using the chemical product to produce the product, such as an intermediate or end product, the user of the end product or the user of the end-of-life product.

The end-of-life instructions 150 may be received by an end-of-life instructions consuming service 210 of an end product user or an end-of-life product user. The end-of-life instructions 150 may be provided by an end-of-life instructions providing service 214 of the chemical product producer, any intermediate product producer using the chemical product to produce the intermediate product or the end product producer using the chemical product to produce the end product.

The end-of-life instructions 150 may be provided as part of a chemical product passport. The chemical product passport may include the decentral identifier associated with the chemical product and/or data related to chemical product as described in the context of FIG. 1. By providing the end-of-life instructions 150 together with the chemical product passport, the end-of-life instructions 150 may be inherently linked to the decentral identifier associated with the chemical product and/or data related to chemical product. This link may be persistent or non-persistent.

If the link to the decentral identifier associated with the chemical product and/or data related to chemical product is persistent the end-of-life instructions 150 associated with the chemical product are linked to the chemical product through the product chain up to the end product and may be accessible through the end product passport via the decentral identifier associated with the chemical product and/or data related to chemical product. An example product passport will be described in FIG. 3. In particular, the end-of-life instructions 150 may be provided in connection with the decentral identifier associated with the chemical product and/or the data related to chemical product and such connection may be maintained on any data exchange relating to the chemical product data. This way the end-of-life instructions 150 may stick directly to the data flow in the decentral network via the decentral identifier associated with the chemical product and/or the data related to the chemical product. Through the digital linking of the decentral identifier associated with the chemical product and the end-of-life instruction(s), the physical use of material and the associated peer-to-peer data exchange enable passing of end-of-life instructions through the production and/or life cycle chain.

If the link to the decentral identifier associated with the chemical product and/or data related to chemical product is non-persistent the relation of the end-of-life instructions 150 to the chemical product can be maintained by linking the end-of-life instructions 150 to any of the decentral identifiers associated with the products produced based on the chemical product and/or data related to the product(s) produced based on the chemical product. In both instances the inherent link to end-of-life instructions and the associated end-of-life treatment of the end product for recovery of the chemical material can be maintained.

FIGS. 3a-c illustrate schematically end-of-life instructions and product passports including the chemical product passport with end-of-life instructions.

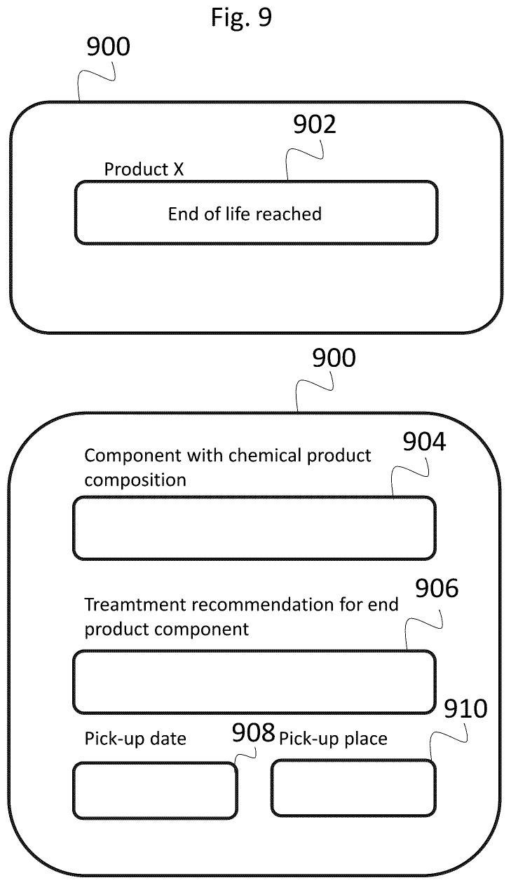

FIG. 3a illustrates end-of-life instructions in form of a simplified JSON file format. The end-of-life instructions may be linked to the decentral identifier associated with the chemical product. The end-of-life instructions may include a specification of the part of the end-of-life product that includes the chemical product. In this case it may be a component of the end-of-life product. The end-of-life instructions may include a specification of the reuse method of the part of the end-of-life product. In this case it may be chemical recycling. The end-of-life instructions may include a specification of the pick-up date of the part of the end-of-life product. In this case it may be a date calculated from the request date or a preset date, e.g. if the end-of-life stage is reached through pre-defined timing. The end-of-life instructions may include a specification of the pickup location of the part of the end-of-life product. In this case it may be a location determined based on the location of the end-of-life product. Through the end-of-life instructions the and-of-life product user upon execution may be provided with the user interface illustrated in FIG. 9.

The product passport configuration shown in FIG. 3b is only one illustrative example and different example configurations for product passports anchored by decentral identifiers exist. Different configurations include different parent, child, grandchild relationships for passports generated in the production chain up to an end product and/or the lifecycle chain up to the end-of life.

FIG. 3b illustrates a fully embedded configuration for different passports generated in the production chain up to the end product. For multiple production stages individual passports may be generated. The passport generation may include the providing of a decentral identifier and an authentication mechanism for each of the multiple production stages. By fully embedding the chemical product passport into the end product passport, the end-of-life instruction may be embedded into the end product passport. This way the end-of-life instruction may be available at any stage of the production chain up to the end product. FIG. 3c illustrated the product passport of FIG. 3b in a simplified JSON file format.

In other embodiments individual configuration for different passports generated in the production chain up to the end product or the lifecycle chain up to end-of life may be used. For multiple stages in the production chain up to the end product or the lifecycle chain up to end-of life individual passports may be generated. The passport generation may include the providing of a decentral identifier and an authentication mechanism for each of the multiple stages. The relationships of passports at different stages of the production chain up to the end product or the lifecycle chain up to end-of life may be based on links between such passports.

In yet other embodiments anchored configuration for different passports generated in the production chain up to the end product or the lifecycle chain up to end-of life may be used. For multiple stages of the production chain up to the end product or the lifecycle chain up to the end-of life individual passports may be generated. The passport generation may include the providing of a decentral identifier and an authentication mechanism for each of the multiple stages of the production chain up to the end product or the lifecycle chain up to end-of life. The relationships of passports at different stages may be anchored to the end product passport based on links from passports at different production stages or lifecycle stages to the end product passport.

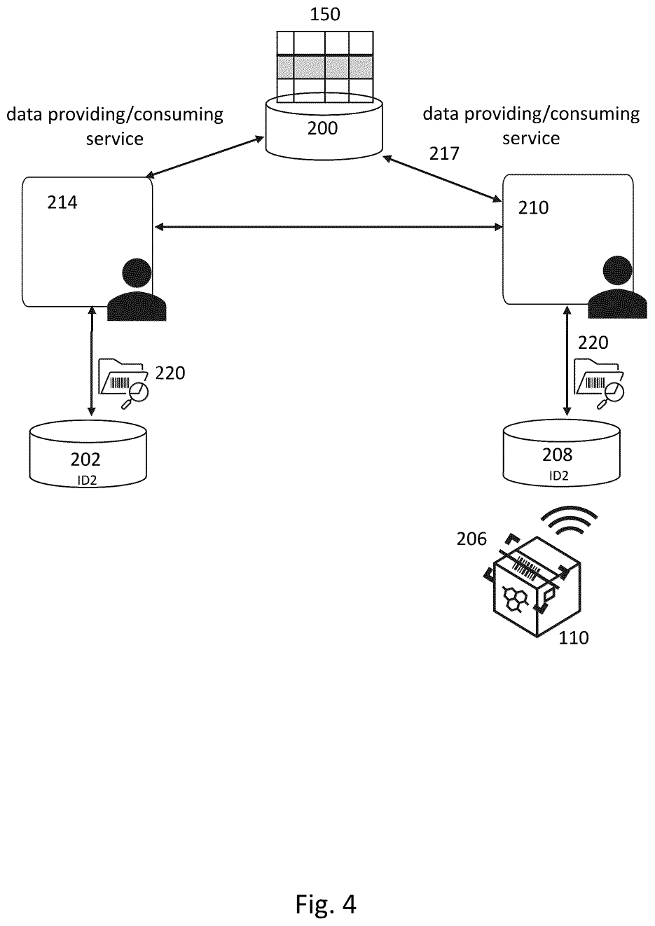

FIG. 4 illustrates schematically another example of a method or apparatus for providing end-of-life instructions for the chemical product used to produce the end product.

The embodiment of FIG. 2 illustrates the providing of the end-of-life instructions 150 through the chemical product passport as explained in the context of FIG. 1. This transfer may occur upon delivery of the chemical product to the intermediate or end product producer. The embodiment of FIG. 4 illustrates the providing of the end-of-life instructions 150 through an end-of-life management system 200. The end-of-life management system 200 may be associated with the data providing service 210 of the user of the end product or the user of the end-of-life product. The end-of-life management system 200 may include a registry providing the end-of-life instructions 150 in relation to the decentral identifier associated with the chemical product. The end-of-life instructions 150 may be provided by the decentral end-of-life application for access by the instruction consuming service 210 of the user of the end product or the user of the end-of-life product. The end-of-life instructions 150 may be provided by the decentral end-of-life application based on the decentral identifier associated with the chemical product or the end product produced from the chemical product.

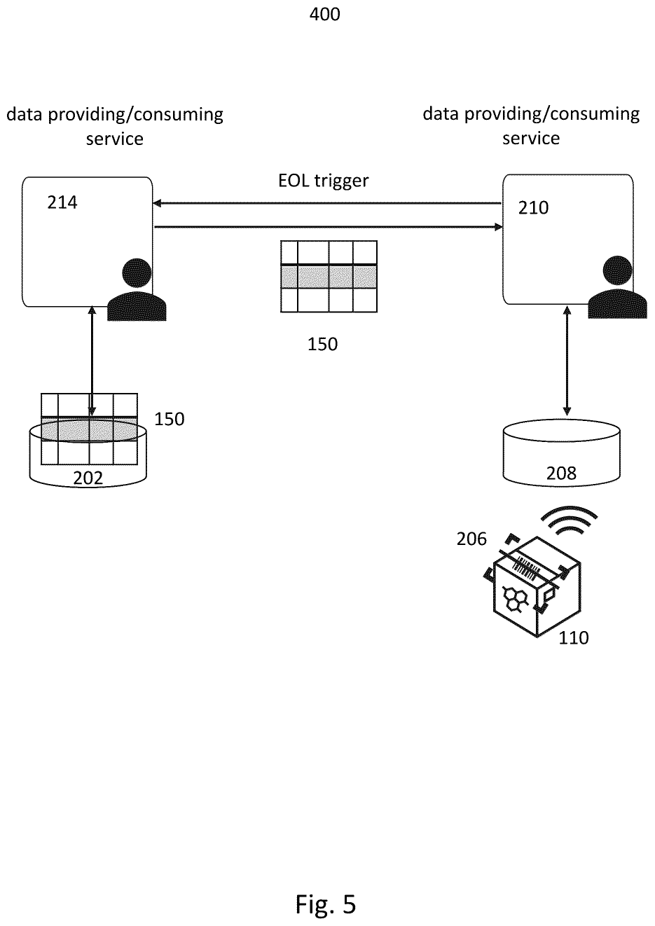

FIG. 5 illustrates schematically another example of a method or apparatus for providing end-of-life instructions for the chemical product used to produce the end product.

The embodiments of FIGS. 2 and 4 illustrate the providing of the end-of-life instructions 150 through the chemical product passport as explained in the context of FIG. 1. In the embodiment of FIG. 5 the end-of-life instructions 150 are provided upon receipt of the end-of-life (EOL) trigger directly by the computing node of the chemical product producer 214.

The chemical product 110 as produced by a chemical production network may be provided in association with a chemical product passport as described in the context of FIG. 1. The chemical product passport may be used to generate the end product passport as described in the context of FIGS. 3a-c for different configurations to trace the chemical product through the production and/or life cycle chain. The end-of-life instructions 150 associated with the chemical product may be stored in a decentral data base 200. The end-of-life instructions 150 may be stored in a data base 202 associated with the data owner, such as the producer of the chemical product 110. The end-of-life instructions 150 may relate to the chemical identifier, in particular the decentral identifier, associated with the chemical product 110.

The chemical product identifier, in particular the decentral identifier, may be accessible via the end product passport to the computing node configured to generate the end-of-life trigger based on the end-of-life event. The end-of-life trigger may be associated with or include data related to the end-of-life event. The data related to the end-of-life instructions 150 may include or be associated with a representation of the end-of-life instructions 150. Such representation may include a pointer to the end-of-life instructions 150 e.g. stored in a decentral data base 202 of the chemical product producer. The data related to the end-of-life instructions may include or relate to authentication and/or authorization information linked to the chemical product identifier, in particular the decentral identifier. The authentication and/or authorization information may be provided for authentication and/or authorization of a data providing service and/or data consuming service 210, 214. The data related to the end-of-life instructions 150 may be fetched from a decentral discovery system or may be provided as part of the chemical product passport.

The end-of-life trigger and the chemical product identifier, in particular the decentral identifier, may be provided to the computing node of the chemical producer 214. Based on the chemical product identifier a request to access the end-of-life instructions 150 related to the chemical product identifier may be triggered by the end-of-life instructions consuming service 210. The chemical product identifier may be provided to the end-of-life instructions providing service 214 associated with or of the producer of the chemical product 110. In addition, authentication and/or authorization information may be provided. The request may be authenticated and/or authorized to access the end-of-life instructions 150 related to the chemical product identifier.

Based on successful authorization and/or authentication access to the end-of-life instructions 150 related to the chemical product identifier may be granted.

For access at least the chemical product identifier may be provided to the end-of-life instructions providing service 214. The end-of-life instructions providing service 214 may use the received chemical product identifier to retrieve the end-of-life instructions associated with the chemical product 110 from the decentral database 202. The end-of-life instructions associated with the chemical product 110 provided to the end-of-life instructions providing service 214 may be provided to the end-of-life instructions consuming service 210. The end-of-life instructions associated with the chemical product 110 may be stored in the data base 208 associated with the end-of-life user of the chemical product 110. The end-of-life instructions associated with the chemical product 110 may be executed by the computing node associated with the end-of-life user of the chemical product 110.

In this sense the end-of-life instructions may be provided directly from the chemical producer to the end-of-life user upon end-of-life trigger to ensure most recent end-of-life instructions are provided.

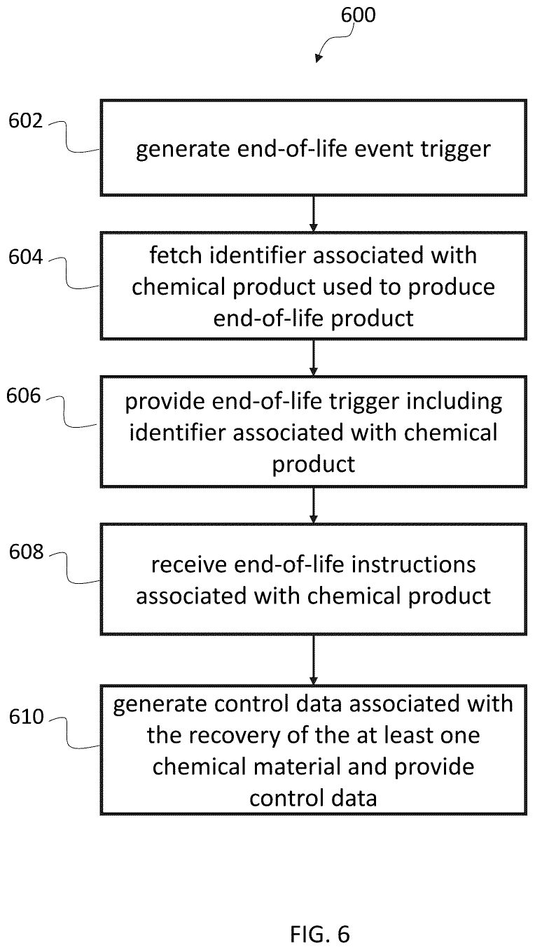

FIG. 6 illustrates schematically a flowchart of a method 600 for controlling one or more end-of-life product(s) to recover at least one chemical material from the one or more end-of-life product(s).

When the end product reaches its end of life, the end product may be disposed by end users via waste collection systems. The waste system may be municipal waste collection or more elaborate waste collection system such as provided by end product return systems. The waste collection system may include sensors such as optical sensors to detect receipt of the end product. Upon return of the end product, the end product may be declared as end-of-life product.

At least one end-of-life trigger may be generated 602 e.g. upon return of the end product by the waste collection system. The end-of-life trigger may relate to registration of the end-of-life product by the waste collection system. The end product may include an end product identifier physically connected to the end product. The waste collection system may provide such identifier by sensor measurement. For example, an optical sensor registers a printed code, such as a QR-code, in which the end product identifier is encoded.

The chemical product identifier associated with chemical product used to produce the end-of-life product may be fetched 604 through the relation to the end product identifier. One way for realizing such a relation via a product passport including the chemical product passport is illustrated in FIG. 3. The end-of-life trigger may relate to or include the end product identifier, particularly the decentral end product identifier. The end product identifier may relate to or include the chemical product identifier associated with the chemical product used to produce the end product. The end-of-life trigger may include or relate to the chemical product identifier, in which case the step of fetching the chemical product identifier is not required.

The end-of-life trigger may be provided 606 to receive 608 end-of-life instructions associated with chemical product. The end-of-life instructions may be provided by the chemical product producer and/or to a consuming service associated with a chemical product producer. The end-of-life instruction associated with the chemical product may be provided based on the end-of-life trigger. The end-of-life instruction may be part of the chemical product passport associated with the chemical product contained in the end product as described in the context of FIGS. 2-4.

The end-of-life instruction may be received 608 based on the chemical product identifier associated with the chemical product contained in the end product as described in the context of FIG. 5. The end-of-life instruction may be related to the chemical product identifier, in particular the decentral. The end-of-life instruction may be stored in a database of the chemical producer for access based on the end-of-life trigger.

The end-of-life trigger may be provided to the end-of-life trigger service and via a peer-to-peer communication to the end-of-life trigger consuming service of the chemical product producer. Upon providing the generated end-of-life trigger, the end-of-life instructions may be accessed by an end-of-life instruction consuming service, particularly transferred to the end-of-life instruction consuming service. Upon receipt of the end-of-life instruction, the end-of-life instruction may be executed to recover the chemical material contained in the end-of-life product.

The end-of-life instruction, when executed by a computing node, may initialize the to recover the chemical material from the end-of-life product. Control data associated with the recovery of the at least one chemical material may be generated and provided 610. The end-of-life instruction may be executed upon generation of the end-of-life trigger and providing of such trigger to the end-of-life instruction. The end-of-life instruction may be executed to initiate the process to recover the chemical material contained in the end-of-life product.

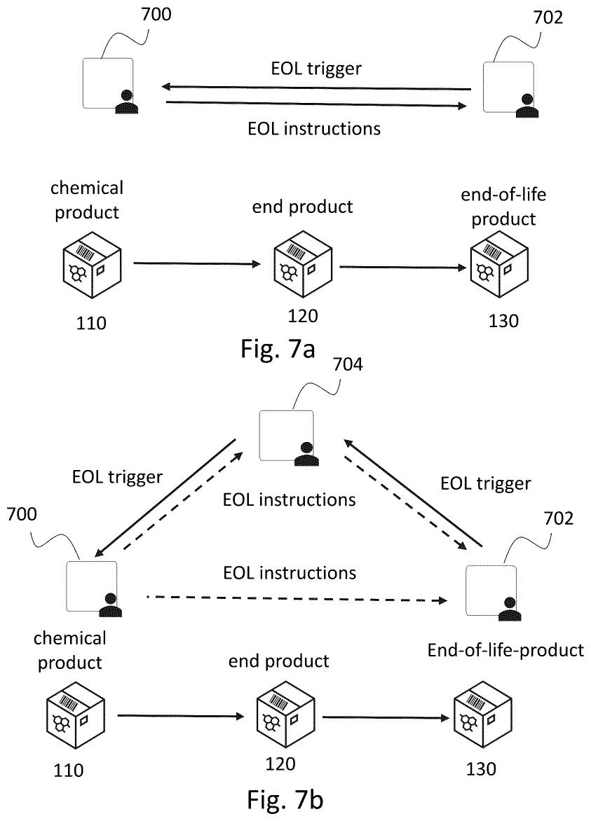

FIGS. 7a, b illustrate schematically the use of end-of life trigger events to execute end-of-life instruction associated with the chemical product.

The end-of-life trigger may be generated based on sensor detection of the end-of-life product 130. The end-of-life (EOL) trigger may be generated based on end-of-life instructions 150 provided with the end product passport, that may be triggered by the end-of-life user. The end-of-life trigger may be generated by any participant node of the decentral network, such as the computing node of the end-of-life user. As shown in FIG. 7a, the participant node 702 triggering the end-of-life event may execute instructions configured to provide the end-of-life trigger to the participant node 700 providing the end-of-life instructions. The participant node 702 generating the end-of-life trigger may act as end-of-life trigger providing service 702. The participant node 702 generating the end-of-life trigger may also receive the end-of-life instructions. The participant node 702 generating the end-of-life trigger may act as end-of-life instructions consuming service 702.

As shown in FIG. 7b, the participant node 702 generating the end-of-life trigger may execute instructions configured to provide the end-of-life trigger to the participant node 704 acting as intermediary to the computing node 700 configured to provide the end-of-life instructions. The participant node 702 generating the end-of-life trigger may act as end-of-life trigger providing service 702. The end-of-life trigger may be provided to the intermediary node 704, which may be configured to provide the end-of-life trigger to the computing node 700 configured to provide the end-of-life instructions. The participant node 702 generating the end-of-life trigger may receive the end-of-life instructions via the intermediary node 704 or directly from the end-of-life instructions providing service 700. The participant node 702 generating the end-of-life trigger may act as end-of-life instructions consuming service 702. The end-of-life instructions may be provided to any computing node acting as end-of-life instructions consuming service 702. The end-of-life instructions may be used to generated control data by the end-of-life consuming service 702.

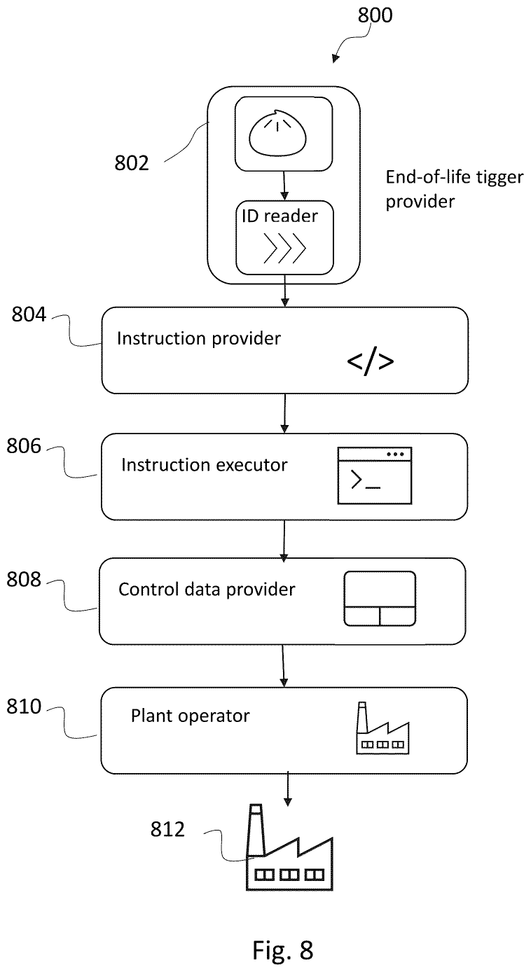

FIG. 8 illustrates schematically examples of methods, systems 800 or apparatuses 802-810 for reusing the end-of-life product or parts thereof to recover the chemical material contained in the end-of-life product.

For reusing the end-of-life product produced based on the chemical product or parts thereof and for recovery of chemical material, the end-of-life triggers and/or end-of-life instructions may be provided via the decentral network. The system components may hence be implemented by different computing node(s) associated with participants of the decentral network. One computing node may implement one or more components of the system 800.

A trigger provider 802 may be configured to generate at least one end-of-life trigger based on the end-of-life event associated with the end-of-life product 130. The end-of-life trigger may be associated with the at least one chemical product and at least one chemical product identifier. The trigger provider 802 may be associated with the user of the end product 120 or the user of the end-of-life product 130.

When the end product 120 reaches its end-of-life, the end product 120 may be disposed by end users via waste collection systems. The waste collection system may be municipal waste collection system or more elaborate waste collection system such as provided by end product return systems. The trigger provider 802 may be implemented by the waste collection system or by the computing node associated with the waste collection system.

The waste collection system may include sensors such as optical sensors to detect receipt of the end product 120. Upon return of the end product 120, the end product 120 may be declared as end-of-life product 130, which may signify the end-of-life event. The end-of-life event may relate to registration of the end-of-life product 130 by the waste collection system. The end-of-life trigger may be generated upon return or registration of the end product by the waste collection system.

The end product 120 may include an end product identifier physically connected to the end product. The waste collection system may provide such identifier by sensor measurement. For example, an optical sensor registers a printed code, such as a QR-code, in which the end product identifier is encoded. The end-of-life trigger may relate to or include the end product identifier, particularly the decentral end product identifier. The end product identifier may be related to the chemical product identifier associated with the chemical product contained in the end product. The end-of-life trigger may include or relate to the chemical product identifier. The chemical product identifier associated with chemical product contained in end-of-life product may be fetched through the relation to the end product identifier. One way for realizing such a relation via a product passport including the chemical product passport is illustrated in FIGS. 3b and c.

The end-of-life trigger may be generated based on the chemical product identifier, particularly the decentral identifier associated with the chemical product. The generated end-of-life trigger may be provided to the end-of-life trigger service and via a peer-to-peer communication to the end-of-life event consuming service of the chemical product producer. Upon providing the generated end-of-life trigger, the end-of-life instructions may be provided to initiate the reuse process to recover the chemical material contained in the end-of-life product.

An instructions provider 804 may be configured to provide, based on at least one end-of-life trigger, at least one end-of-life instruction associated with the chemical product. The generated end-of-life trigger may be provided to fetch the end-of-life instructions provided by the chemical product producer. The end-of-life trigger may relate to a decentral identifier associated with the chemical product used to produce the end-of-life product. The end-of-life instruction may be provided in relation to the decentral identifier associated with the chemical product used to produce the end-of-life product.

The end-of-life instructions may be part of the chemical product passport as described in the context of FIGS. 1, 2, 3a-c, 4 and 5. Through the relation of the chemical product identifier, particularly the decentral identifier, with the end product identifier, the end-of-life instructions may be accessible by the user of the end product or the user of the end-of-life product.

The end-of-life instructions may be stored in a decentral data base e.g. associated with the chemical product producer. The end-of-life instructions may be provided in response to providing the generated end-of-life trigger. The end-of-life instructions may be provided via the decentral network from the chemical producer. The end-of-life trigger may be provided by an end-of-life trigger service of the end product user or the end-of-life product user. The end-of-life trigger may be received by the end-of-life event consuming service of the chemical product producer.

The end-of-life instruction may be provided by the end-of-life instruction providing service of the chemical product producer. The end-of-life instruction may be received by the end-of-life instruction consuming service of the end product user or the end-of-life product user.

The generated end-of-life trigger may be provided to a trigger consuming service associated with a chemical product producer. Through the relation of the chemical product identifier, particularly the decentral identifier, with the end product identifier, the chemical product identifier may be provided to the trigger consuming service associated with the chemical product producer.

The end-of-life trigger may relate to or include the decentral identifier associated with the end-of-life product and/or data related to end-of-life product data to provide access to end-of-life product data for the chemical producer. This way end-of-life product data may be provided to the chemical producer. Such end-of-life product data may include properties of the end-of-life product related to the performance or use of the chemical product contained in the end-of-life product. The end-of-life instruction providing service associated with the chemical product producer may provide or transfer the end-of-life instruction related to the chemical product identifier, particularly the decentral identifier, to the end-of-life instruction consuming service associated with the user of the end product 120 or the user of the end-of-life product 130. Upon receipt the end-of-life instructions may be initialized and executed to to recover the chemical material.

An instructions executor 806 may be configured to generate, based on at least one end-of-life instruction, control data associated with the recovery of the at least one chemical material from the one or more end-of-life product(s) produced from at least one chemical product. The instruction executor 806 may be configured to execute the at least one end-of-life instruction and to initialize the treatment of the end-of-life product for recovery of the chemical material. The end-of-life instruction, when executed by a computing node, may initialize the process for recovery of the chemical material. The end-of-life instruction associated with the chemical product may include or generate control data for sorting and/or collecting the end-of-life product. The end-of-life instruction associated with the chemical product may relate to or generate control data for providing the end-of-life product or at least one part of the end-of-life product to a chemical treatment plant.

A control data provider 808 may be configured to provide the control data to initialize the process for recovery of the chemical material. The control data may be associated with one or more process step(s) of the reuse process. The reuse process may include returning end-of-life product(s) or parts thereof to the chemical product producer and/or treating end-of-life product(s) or parts thereof by the chemical product producer. The control data may relate to the chemical and/or physical treatment of the end-of-life product to recover the chemical material.

The control data may relate to chemical and/or physical treatment method(s) suitable for treating the end-of-life product or part(s) thereof to recover the chemical material. The control data may relate to chemical and/or physical treatment method(s) to be conducted by the chemical product producer. The control data may relate to a return of the end-of-life product or part(s) thereof to the chemical product producer. The control data may relate to chemical and/or physical treatment plant(s) suitable for treating the end-of-life product or part(s) thereof to recover the chemical material. The control data may relate to chemical and/or physical treatment plant(s) suitable for recovering the chemical material from the end-of-life product or part(s) thereof. The control data may relate to collecting, sorting and/or transporting of end-of-life products or part(s) thereof to recover the chemical material. The control data may relate to providing the end-of-life product or at least one part of the end-of-life product to a chemical and/or physical treatment plant associated with a chemical product producer.

A plant operator 810 of the chemical and/or physical treatment plant 812 may be configured to receive the control data and to initialize the treatment of the end-of-life product or parts thereof to recover the chemical material. The operator of the chemical and/or physical treatment plant may be associated with the chemical product producer producing the chemical product.

The chemical and/or physical treatment plant 812 for treating the end-of-life product or part(s) thereof to recover the chemical material may be associated with the chemical product producer.

The chemical and/or physical treatment plant 812 may physically execute the treatment of the end-of-life product or parts thereof to recover the chemical material.

FIG. 9 illustrates schematically a user interface for initiating the process to treat the end-of-life product and to recover chemical material.

The user interface 900 illustrates two views the end-of-life product user may get to trigger the end-of-life event and in response to triggering the end-of-life event. The upper view illustrates the view for triggering the end-of-life event. For instance, based on reading the QR code of the end product, the user may be prompted to a screen specifying the product and an end-of-life reached button 902. Upon clicking the end-of-life reached button 902, the end-of-life trigger may be generated, and the end-of-life instructions may be retrieved as described in the context of FIGS. 1 to 9. The end-of-life instruction may be configured to generate control data for collecting the end-of-life product. The end-of-life instructions may specify, as illustrated in FIG. 3a, the component of the end-of-life product containing the chemical product 904, the re-use or treatment method 906, the date 908 and location 910 for collecting the end-of-life product or the respective component may be displayed. This way the end-of-life instructions may be retrieved to generate control data related to the process step of collecting the end-of-life product or the component of the end-of-life product.

The end-of life instruction may include further information related to further process steps. For instance, the end-of-life instruction may relate to the re-use method. The end-of-life instruction may be configured to generate control data for collecting, sorting, transporting and/or treating the end-of-life product or at least one part of the end-of-life product. The end-of-life instruction may be configured to generate control data for providing the end-of-life product or at least one part of the end-of-life product to a chemical and/or physical treatment plant associated with the chemical product producer. The control data may relate to the return of the end-of-life product or part(s) thereof to the chemical product producer. The control data may relate to chemical and/or physical treatment method(s) suitable for treating the end-of-life product or part(s) thereof to recover the chemical material. The control data may relate to chemical and/or physical treatment method(s) to be conducted by the chemical product producer.

The present disclosure has been described in conjunction with preferred embodiments and examples as well. However, other variations can be understood and effected by those persons skilled in the art and practicing the claimed invention, from the studies of the drawings, this disclosure and the claims.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

Any disclosure and embodiments described herein relate to the methods, the systems, devices, the computer program element lined out above and vice versa. Advantageously, the benefits provided by any of the embodiments and examples equally apply to all other embodiments and examples and vice versa.

All terms and definitions used herein are understood broadly and have their general meaning.

1. A method for controlling one or more end-of-life product(s) to recover at least one chemical material from the one or more end-of-life product(s), wherein the one or more end-of-life product(s) are produced based on at least one chemical product, the method comprising the steps:
providing, based on at least one end-of-life trigger, at least one end-of-life instruction associated with the chemical product, wherein the at least one end-of-life trigger is associated with the at least one chemical product and at least one chemical product identifier;
generating, based the at least one end-of-life instruction, control data associated with the recovery of the at least one chemical material from the one or more end-of-life product(s) produced based on at least one chemical product; and
providing the control data to initialize a process for recovery of the chemical material.

2. The method of claim 1, wherein the chemical material includes one or more compound(s) produced from the end-of-life product or part(s) thereof, wherein the chemical material includes one or more compound(s) suitable for producing the at least one chemical product.

3. The method of claim 1, wherein the at least one end-of-life trigger is associated with at least one chemical product identifier associated with the chemical product used to produce the end-of-life product.

4. The method of claim 1, wherein the at least one end-of-life instruction associated with the chemical product used to produce the end-of-life product is provided in relation to the at least one chemical product identifier associated with the chemical product used to produce the end-of-life product.

5. The method of claim 1, wherein the at least one end-of-life instruction associated with the chemical product is provided in response to providing the end-of-life trigger to an instructions providing service associated with an end product producer, an end product user or an end-of-life product user, wherein the at least one end-of-life instruction associated with the chemical product is provided via the decentral network from the instructions providing service associated with the end product producer, the end product user or the end-of-life product user.

6. The method of claim 1, wherein the at least one end-of-life instruction associated with the chemical product is provided in response to providing the end-of-life trigger to an instructions providing service associated with a chemical product producer, wherein the at least one end-of-life instruction associated with the chemical product is provided via the decentral network from the instructions providing service associated with the chemical product producer.

7. The method of claim 1, wherein the at least one end-of-life instruction associated with the chemical product is configured to generate control data for providing the end-of-life product or at least one part of the end-of-life product to a chemical and/or physical treatment plant associated with the chemical product producer.

8. The method of claim 1, wherein the control data associated with one or more process step(s) of the process are generated, wherein the process includes returning end-of-life product(s) or parts thereof to the chemical product producer and/or treating end-of-life product(s) or parts thereof by the chemical product producer.

9. The method of claim 1, wherein the control data relates to a chemical and/or physical treatment of the end-of-life product to recover the chemical material.

10. The method of claim 1, wherein the control data relates to chemical and/or physical treatment method(s) suitable for treating the end-of-life product or part(s) thereof to recover the chemical material.

11. The method of claim 1, wherein the control data relates to chemical and/or physical treatment method(s) to be conducted by the chemical product producer, wherein the control data relates to a return of the end-of-life product or part(s) thereof to the chemical product producer.

12. An apparatus for controlling one or more end-of-life product(s) to recover at least one chemical material from the one or more end-of-life product(s), wherein the one or more end-of-life product(s) are produced based on at least one chemical product, the apparatus being configured to:
providing, based on at least one end-of-life trigger, at least one end-of-life instruction associated with the chemical product, wherein the at least one end-of-life trigger is associated with the at least one chemical product and at least one chemical product identifier;
generating, based on at least one end-of-life instruction, control data associated with the recovery of the at least one chemical material from the one or more end-of-life product(s) produced based on at least one chemical product; and
providing the control data to initialize a process for recovering the chemical material.

13. A system for controlling one or more end-of-life product(s) to recover at least one chemical material from the one or more end-of-life product(s), the system comprising:
the apparatus for controlling one or more end-of-life product(s) to recover at least one chemical material from the one or more end-of-life product(s) of claim 12, and
a chemical and/or physical treatment plant for treating the end-of-life product or part thereof to recover the chemical material, wherein the chemical and/or physical treatment plant is associated with the chemical product producer.

14. A chemical product associated with at least one end-of-life instruction configured to control one or more end-of-life product(s) to recover at least one chemical material from the one or more end-of-life product(s), wherein the end-of-life instructions are configured to provide control data according to the method of claim 1.

15. A method for recovering chemical material and/or to refeed chemical material into one or more chemical production process(es) comprising using the control data provided according to the method of claim 1.
